(19) United States
(12) Patent Application Publication
(10) Pub. No.: US 2025/0041263 A1
(43) Pub. Date: Feb. 6, 2025

(54) USE OF CATECHIN FOR THE TREATMENT OF FGFR-RELATED BONE REPAIR AND BONE FORMATION IMPAIRMENT

(71) Applicants: INSTITUT NATIONAL DE LA SANTÉ ET DE LA RECHERCHE MÉDICALE, Paris (FR); UNIVERSITÉ PARIS CITÉ, Paris (FR); FONDATION IMAGINE, Paris (FR); ASSISTANCE PUBLIQUE-HÔPITAUX DE PARIS (APHP), Paris (FR)

(72) Inventors: Laurence LEGEAI-MALLET, Paris (FR); Anne MORICE, Paris (FR)

(21) Appl. No.: 18/717,719

(22) PCT Filed: Dec. 19, 2022
(86) PCT No.: PCT/EP2022/086563

§ 371 Date: Jun. 7, 2024

(30) Foreign Application Priority Data
Dec. 20, 2021 (EP) 21306858.8

(51) Int. Cl. A61K 31/353; A61P 19/08

(52) U.S. Cl. CPC A61K 31/353; A61P 19/08

(57) ABSTRACT

The present invention relates to a method for the treatment of FGFR-related bone repair and bone formation and quality impairment. The inventors provide data confirming that abnormal activation of the FGFR3 signaling impairs the bone formation and repair process in HCH mandible characterized by the presence of pseudarthrosis in many calluses and bone structure similar to osteoporotic bones. Interestingly, the treatment with catechin partially restore the defective bone formation and repair. The present invention thus relates to a method for the treatment of FGFR-related bone repair and bone formation in a subject in need thereof comprising administering to the subject a therapeutically effective amount of at least one catechin.

### FIELD OF THE INVENTION

The present invention relates to methods and pharmaceutical compositions for the treatment of FGFR-related bone repair and bone formation and quality impairment.

### BACKGROUND OF THE INVENTION

The Fibroblast Growth Factor Receptor (FGFR) are key genes involved in bone formation. Patients with craniosynostosis and chondrodysplasia, linked to FGFRs gain-of-function mutations, present craniofacial and mandibular malformations (Kolar JC 2017). FGFR2-related craniostenosis (e.g. Crouzon, Apert, and Pfeiffer syndromes) are characterized by the presence of uni- or bicoronal craniosynostosis with fusion of one or more cranial sutures, resulting in variable cranial deformities with hypertelorism, exorbitism, hypoplasia of the midface and prognathism. FGFR3-related craniosynostosis (e.g. Muenke syndrome and Crouzon syndrome and acanthosis nigricans) display craniofacial anomalies namely uni or bicoronal craniosynostosis, and midface hypoplasia. FGFR3-related chondrodysplasia such as achondroplasia, Severe Achondroplasia with developmental delay and acanthosis nigricans (SADDAN) and hypochondroplasia (HCH) are characterized by short limbs, skull base anomalies, macrocephaly, deafness, hypoplasia of the midface and prognathism. Maxillofacial and neurosurgical surgeries is indicated for these patients with craniosynostosis and chondrodysplasia in order to correct craniofacial and skull anomalies. The process of bone healing occurs in two different ways depending on mechanical stability. Stable fractures healing occurs via membranous ossification. In contrast, the healing of unstable fracture is under the control of endochondral ossification. In this case, a large cartilage template forms in the fracture gap that is replaced by bone to bridge the two ends of broken bone.

Fracture healing is a complex process involving a cascade of cellular events that include the initial bleeding and inflammation, recruitment and proliferation of mesenchymal cells, subsequent formation of cartilaginous callus and its gradual replacement by bony callus. A variety of growth factors/cytokines regulate skeletal development and homeostasis and can also regulate the fracture healing. It is well known that receptor tyrosine kinase (RTK) plays a role in bone repair. Among RTK, FGFR3 regulates the cartilaginous callus formation and replacement by bone. In a mouse model of chondrodysplasia (Fgfr3^{Y367C/+}) FGFR3 gain-of function mutation impairs bone regeneration in non-stabilized tibial fractures and induces a pseudarthrosis phenotype in the callus (Julien et al 2020).

The understanding of skeletal repair and bone formation is essential for developing therapies to be used to improve bone healing after surgical osteotomies or traumatic bone fractures. The exact function of FGFRs in mandible formation and repair remains to be understood. In the maxillofacial skeleton, the mandible is the largest and strongest bone of the face, the abnormal growth development contributes to maxillo-mandibular imbalance and subsequent dento-occlusal anomalies and facial growth perturbation. Two processes of ossification control the mandibular development 1) endochondral ossification regulates the condylar and Meckel cartilage formation and 2) the membranous ossification controls the ramus formation and elongation.

In order to investigate the abnormal FGF signaling during mandibular bone formation and repair, the inventors studied the HCH mouse model (Fgfr3^{N534K/+}). The HCH mouse model displays the HCH maxillofacial features namely macrocephaly and prognathism. Non-stabilized fractures of the mandibles of adult HCH mice were performed and the formation of the callus repair was analyzed at various key points of the reparation. The inventor's data confirm that abnormal activation of the FGFR3 signaling impairs bone formation and resorption, bone quality and bone repair process in HCH mandible characterized by the presence of pseudarthrosis in many calluses. Interestingly, the treatment with the phenol compound catechin isolated from Theobroma cacao partially restores the defective bone reparation of the callus, increases the bone volume/total volume without pseudarthrosis.

### SUMMARY OF THE INVENTION

The present invention relates to methods and pharmaceutical compositions for the treatment of FGFR-related bone repair and bone formation and quality impairment. In particular, the present invention is defined by the claims.

### DETAILED DESCRIPTION OF THE INVENTION

The present invention relates to a method of treatment of FGFR-related bone repair and bone formation impairment in a subject in need thereof comprising administering to the subject a therapeutically effective amount of at least one catechin.

The present invention also relates to a method of restoring defective bone reparation in a subject in need thereof comprising administering to the subject a therapeutically effective amount of at least one catechin.

As used herein, the term “subject” or “patient” denotes a mammal, such as a rodent, a feline, a canine, and a primate. Particularly, the subject according to the invention is a human. Particularly, the subject according to the invention is an adult. Particularly, the subject according to the invention is a child, a teenager, or an elderly persons. In some embodiments, the patient is less than 15 years old. In some embodiments, the patient is less than 10 years old. In some embodiments, the patient is less than 7 years old. In some embodiments, the patient is less than 5 years old. In some embodiments, the patient is less than 3 years old. In some embodiments, the patient is an adult. In some embodiments, the subject is more than 15 years old. In some embodiments, the subject is more than 20 years old. In some embodiments, the subject is more than 25 years old. In some embodiments, the subject is more than 30 years old. In some embodiments, the subject is more than 35 years old.

As used herein, the term “bone” refers to a rigid tissue that constitutes part of the skeleton in most vertebrate animals. Bones protect the various organs of the body, produce red and white blood cells, store minerals, provide structure and support for the body, and enable mobility. Bones come in a variety of shapes and sizes and have a complex internal and external structure. They are lightweight yet strong and hard, and serve multiple functions. Bone tissue (osseous tissue) is a hard tissue, a type of specialized connective tissue. It has a honeycomb-like matrix internally, which helps to give the bone rigidity. Bone tissue is made up of different types of bone cells. Osteoblasts and osteocytes are involved in the formation and mineralization of bone; osteoclasts are involved in the resorption of bone tissue. Modified (flattened) osteoblasts become the lining cells that form a protective layer on the bone surface. The mineralized matrix of bone tissue has an organic component of mainly collagen called ossein and an inorganic component of bone mineral made up of various salts. Bone tissue is a mineralized tissue of two types, cortical bone and cancellous (also called trabecular) bone. Other types of tissue found in bones include bone marrow, endosteum, periosteum, nerves, blood vessels growth plate and articular cartilage.

As used herein, the terms “bone formation”, “osteogenesis” or “ossification” relate to the process of bone formation. After progenitor cells form osteoblastic lines, they proceed with three stages of development of cell differentiation, called proliferation, maturation of matrix, and mineralization. Based on its embryological origin, there are two types of ossification, called intramembranous ossification that occurs in mesenchymal cells that differentiate into osteoblast in the ossification center directly without prior cartilage formation and endochondral ossification in which bone tissue mineralization is formed through cartilage formation first. In intramembranous ossification, bone development occurs directly. In this process, mesenchymal cells proliferate into areas that have high vascularization in embryonic connective tissue in the formation of cell condensation or primary ossification centers. This cell will synthesize bone matrix in the periphery and the mesenchymal cells continue to differentiate into osteoblasts. After that, the bone will be reshaped and replaced by mature lamellar bone. Endochondral ossification will form the center of primary ossification, and the cartilage extends by proliferation of chondrocytes and deposition of cartilage matrix. After this formation, chondrocytes in the central region of the cartilage start to proceed with maturation into hypertrophic chondrocytes. After the primary ossification center is formed, the marrow cavity begins to expand toward the epiphysis. Then the subsequent stages of endochondral ossification will take place in several zones of the bone.

In some embodiment, the treatment of the present invention improves bone healing after surgical osteotomies or traumatic bone fractures.

As used herein, the term “surgical osteotomies” refers to a procedure in which a surgeon removes, or sometimes adds, a wedge of bone near a damaged joint. This shifts weight from an area where there is damaged cartilage to an area where there is more or healthier cartilage. As used herein, the term “bone healing” or “fracture healing” or “bone repair” have their general meaning in the art and refer to a proliferative physiological process in which the body facilitates the repair of a bone fracture. The bone healing process has three overlapping stages
1. Inflammation starts immediately after the bone is fractured and lasts for several days. When the bone is fractured, there is bleeding into the area, leading to inflammation and clotting of blood at the fracture site. This provides the initial structural stability and framework for producing new bone.
2. Bone production begins when the clotted blood formed by inflammation is replaced with fibrous tissue and cartilage (known as soft callus). As healing progresses, the soft callus is replaced with hard bone (known as hard callus), which is visible on x-rays several weeks after the fracture.
3. Bone remodeling, the final phase of bone healing, goes on for several months. In remodeling, bone continues to form and becomes compact, returning to its original shape. In addition, blood circulation in the area improves. Once adequate bone healing has occurred, weightbearing (such as standing or walking) encourages bone remodeling.

In some embodiment, the subject suffers from a bone fracture. As used herein, the term “bone fracture” refers to a medical condition in which there is a partial or complete break in the continuity of a bone. In more severe cases, the bone may be broken into several pieces.

In some embodiment, the at least one catechin of the present invention increases the bone density or the bone volume/Total volume (BV/TV). As used herein, the term “bone density”, or “bone volume/Total volume” (BV/TV), is the amount of bone mineral in bone tissue.

In some embodiment, the at least one catechin of the present invention can be used to treat osteoporosis. As used herein, the term “osteoporosis” is a bone disease characterized by deterioration of bone tissue/bone density, which causes bones to become very thin and brittle over time.

As used herein, the term “craniofacial anomalies” refers to diverse group of deformities in the growth of the head and facial bones. Anomaly refers to a medical term meaning “irregularity” or “different from normal.” These abnormalities are present at birth (congenital) and there are numerous variations. Some are mild and some are severe and need surgery.

As used herein, the term “craniosynostosis” refers to a condition in which one or more of the fibrous sutures in a young infant's skull prematurely fuses by turning into bone (ossification), thereby changing the growth pattern of the skull. It can be associated with growth abnormalities of the facial skeleton, known as facio-cranio-stenosis) or even come within the framework of polymalformative syndromes.

As used herein, the term “mandible”, also known as lower jaw or jawbone is the largest, strongest and lowest bone in the human facial skeleton. It forms the lower jaw and holds the lower teeth in place. The mandible sits beneath the maxilla. It is the only movable bone of the skull (discounting the ossicles of the middle ear). It is connected to the temporal bones by the temporomandibular joints.

In some embodiment, the subject has or will suffer from a FGFR-related bone repair and bone formation impairment. In some embodiment, the subject harbours a FGFR gain-of-function mutation.

As used herein, the term “FGFR-related bone repair and bone formation and quality impairment” or “FGFR-related bone repair and bone formation impairment” refer to deficiencies procedure in the bone repair and the bone formation. FGFR-related bone repair impairment refers to a defective bone reparation.

As used herein, the term “Fibroblast growth factors” (FGF) relates a family of cell signalling proteins; they are involved in a wide variety of processes, most notably as crucial elements for normal development in animal cells. Any irregularities in their function lead to a range of developmental defects. These growth factors typically act as systemic or locally circulating molecules of extracellular origin that activate cell surface receptors. A defining property of FGFs is that they bind to a co-receptor heparin and to heparan sulfate. Thus, some are sequestered in the extracellular matrix of tissues that contains heparan sulfate proteoglycans and are released locally upon injury or tissue remodeling.

As used herein, the term “Fibroblast growth factor receptors” (FGFR) relates receptors that bind to members of the fibroblast growth factor (FGF) family of proteins. Some of these receptors are involved in pathological conditions. Distinct membrane FGFR have been identified in vertebrates and all of them belong to the tyrosine kinase superfamily: FGFR1 (see also Fibroblast growth factor receptor 1) (=CD331), FGFR2 (see also Fibroblast growth factor receptor 2) (=CD332), FGFR3 (see also Fibroblast growth factor receptor 3) (=CD333), FGFR4 (see also Fibroblast growth factor receptor 4) (=CD334), FGFRL1 (see also Fibroblast growth factor receptor-like 1) and FGFR6.

In some embodiment, the subject harbours a FGFR3 gain-of-function mutation.

As used herein, the terms “FGFR3”, “FGFR3 tyrosine kinase receptor” and “FGFR3 receptor” are used interchangeably throughout the specification and refer to all of the naturally-occurring isoforms of FGFR3. An exemplary human amino acid sequence of FGFR3 is represented by SEQ ID NO:1.

>sp|P22607|FGFR3_HUMAN Fibroblast growth factor receptor 3
OS = Homo sapiens OX = 9606 GN = FGFR3 PE = 1 SV = 1
SEQ ID NO: 1
MGAPACALALCVAVAIVAGASSESLGTEQRVVGRAAEVPGPEPGQQEQLVFGSGDAVELS
CPPPGGGPMGPTVWVKDGTGLVPSERVLVGPQRLQVLNASHEDSGAYSCRQRLTQRVLCH
FSVRVTDAPSSGDDEDGEDEAEDTGVDTGAPYWTRPERMDKKLLAVPAANTVRFRCPAAG
NPTPSISWLKNGREFRGEHRIGGIKLRHQQWSLVMESVVPSDRGNYTCVVENKFGSIRQT
YTLDVLERSPHRPILQAGLPANQTAVLGSDVEFHCKVYSDAQPHIQWLKHVEVNGSKVGP
DGTPYVTVLKTAGANTTDKELEVLSLHNVTFEDAGEYTCLAGNSIGFSHHSAWLVVLPAE
EELVEADEAGSVYAGILSYGVGFFLFILVVAAVTLCRLRSPPKKGLGSPTVHKISRFPLK
RQVSLESNASMSSNTPLVRIARLSSGEGPTLANVSELELPADPKWELSRARLTLGKPLGE
GCFGQVVMAEAIGIDKDRAAKPVTVAVKMLKDDATDKDLSDLVSEMEMMKMIGKHKNIIN
LLGACTQGGPLYVLVEYAAKGNLREFLRARRPPGLDYSFDTCKPPEEQLTFKDLVSCAYQ
VARGMEYLASQKCIHRDLAARNVLVTEDNVMKIADFGLARDVHNLDYYKKTTNGRLPVKW
MAPEALFDRVYTHQSDVWSFGVLLWEIFTLGGSPYPGIPVEELFKLLKEGHRMDKPANCT
HDLYMIMRECWHAAPSQRPTFKQLVEDLDRVLTVTSTDEYLDLSAPFEQYSPGGQDTPSS
SSSGDDSVFAHDLLPPAPPSSGGSRT

As used herein, the expressions “FGFR3 gain-of-function mutation”, “constitutively active FGFR3 receptor variant”, “constitutively active mutant of the FGFR3” or “mutant FGFR3 displaying a constitutive activity” are used interchangeably and refer to a mutant of said receptor exhibiting a biological activity (i.e. triggering downstream signaling), and/or exhibiting a biological activity which is higher than the biological activity of the corresponding wild-type receptor in the presence of FGF ligand. A constitutively active FGFR3 variant according to the invention is in particular chosen from the group consisting of (residues are numbered according to their position in the precursor of fibroblast growth factor receptor 3 isoform 1-806 amino acids long-): a mutant wherein the serine residue at position 84 is substituted with lysine (named herein below S84L); a mutant wherein the arginine residue at position 200 is substituted with cysteine (named herein below R200C); a mutant wherein the arginine residue at position 248 is substituted with cysteine (named herein below R248C); a mutant wherein the serine residue at position 249 is substituted with cysteine (named herein below S249C); a mutant wherein the proline residue at position 250 is substituted with arginine (named herein below P250R); a mutant wherein the asparagine residue at position 262 is substituted with histidine (named herein below N262H); a mutant wherein the glycine residue at position 268 is substituted with cysteine (named herein below G268C); a mutant wherein the tyrosine residue at position 278 is substituted with cysteine (named herein below Y278C); a mutant wherein the serine residue at position 279 is substituted with cysteine (named herein below S279C); a mutant wherein the glycine residue at position 370 is substituted with cysteine (named herein below G370C); a mutant wherein the serine residue at position 371 is substituted with cysteine (named herein below S371C); a mutant wherein the tyrosine residue at position 373 is substituted with cysteine (named herein below Y373C); a mutant wherein the glycine residue at position 380 is substituted with arginine (named herein below G380R); a mutant wherein the valine residue at position 381 is substituted with glutamate (named herein below V381E); a mutant wherein the alanine residue at position 391 is substituted with glutamate (named herein below A391E); a mutant wherein the asparagine residue at position 540 is substituted with Lysine (named herein below N540K); a mutant wherein the termination codon is eliminated due to base substitutions, in particular the mutant wherein the termination codon is mutated in an arginine, cysteine, glycine, serine or tryptophane codon (named herein below X807R, X807C, X807G, X807S and X807W, respectively); a mutant wherein the lysine residue at position 650 is substituted with another residue, in particular with methionine, glutamate, asparagine or glutamine (named herein below K650M, K650E, K650N and K650Q); a mutant wherein the methionine residue at position 528 is substituted with isoleucine (named herein below M528I); a mutant wherein the isoleucine residue at position 538 is substituted with valine (named herein below 1538V); a mutant wherein the asparagine residue at position 540 is substituted with serine (named herein below N540S); a mutant wherein the asparagine residue at position 540 is substituted with threonine (named herein below N540T). Typically, a constitutively active FGFR3 variant according to the invention is N540K, K650N, K650Q, M528I, 1538V, N540S, N540T or A391E mutant.

In some embodiment, the subject suffers from a FGFR3-related skeletal disease.

As used herein the term “FGFR3-related skeletal disease” is intended to mean a skeletal disease that is caused by an abnormal increased activation of FGFR3, in particular by expression of a constitutively active mutant of the FGFR3 receptor, in particular a constitutively active mutant of the FGFR3 receptor as described above.

In some embodiment, the FGFR3-related skeletal diseases are preferably FGFR3-related chondrodysplasias and FGFR3-related craniosynostosis.

As used herein “FGFR3-related chondrodysplasias” include but are not limited to dwarfism such as hypochondroplasia (HCH), thanatophoric dysplasia (TD) type I, thanatophoric dysplasia type II, achondroplasia (ACH) and SADDAN (severe achondroplasia with developmental delay and acanthosis nigricans).

In particular, the FGFR3-related skeletal disease is dwarfism.

As used herein, the term “dwarfism” has its general meaning in the art and refers to a short stature that results from a genetic or medical condition. Dwarfism is generally defined as an adult height of 147 centimeters or less.

In particular, the FGFR3-related skeletal disease is hypochondroplasia (HCH).

As used herein, the term “hypochondroplasia” (HCH) has its general meaning in the art and relates to a disproportionately short stature, rhizomelia and a head that appears large in comparison with the underdeveloped portions of the body.

In some embodiment, the FGFR3-related chondrodysplasias is a hypochondroplasia caused by expression of the N540K, K650N, K650Q, M528I, 1538V, N540S or N540T constitutively active mutant of the FGFR3 receptor.

In particular, the FGFR3-related skeletal disease is achondroplasia (ACH).

As used herein, the term “achondroplasia” (ACH) has its general meaning in the art and relates to a genetic deficit in which the arms and legs are short, while the torso is typically of normal length and with an enlarged head and prominent forehead.

In particular, the FGFR3-related skeletal disease is thanatophoric dysplasia (TD).

As used herein, the term “thanatophoric dysplasia” (TD) has its general meaning in the art and relates to a severe skeletal deficit characterized by a disproportionately small ribcage, extremely short limbs and folds of extra skin on the arms and legs.

In some embodiment, the FGFR3-related skeletal disease is FGFR3-related craniosynostosis. In some embodiments, the FGFR3-related craniosynostosis corresponds to an inherited or to a sporadic disease.

In particular, the FGFR3-related craniosynostosis is Muenke syndrome caused by expression of the P250R constitutively active mutant of the FGFR3 receptor.

In particular, the FGFR3-related craniosynostosis is Crouzon syndrome with acanthosis nigricans (CAN) caused by expression of the A391E constitutively active mutant of the FGFR3 receptor.

As used herein, the term “craniosynostosis” has its general meaning in the art and relates a condition in which one or more of the fibrous sutures a subject skull prematurely fuses by turning into bone (ossification), thereby changing the growth pattern of the skull. “Crouzon syndrome with acanthosis nigricans” (CAN) is a very rare craniosynostosis.

As used herein, the term “acanthosis nigricans” relates to a brown to black, poorly defined, velvety hyperpigmentation of the skin.

In particular, the subject suffers from a FGFR2 gain-of-function mutation.

As used herein, the term “FGFR2”, “FGFR2 tyrosine kinase receptor” and “FGFR2 receptor” are used interchangeably throughout the specification and refer to all of the naturally-occurring isoforms of FGFR2.

As used herein, the expressions “FGFR2 gain-of-function mutation”, “constitutively active FGFR2 receptor variant”, “constitutively active mutant of the FGFR2” or “mutant FGFR2 displaying a constitutive activity” are used interchangeably and refer to a mutant of said receptor exhibiting a biological activity (i.e. triggering downstream signaling), and/or exhibiting a biological activity which is higher than the biological activity of the corresponding wild-type receptor in the presence of FGF ligand. A constitutively active FGFR2 variant according to the invention is in particular chosen from the group consisting of (residues are numbered according to their position in the precursor of fibroblast growth factor receptor 3 isoform 1-806 amino acids long-). In particular mutations of FGFR2 include: a mutant wherein the tryptophane residue at position 290 is substituted with cysteine (named herein below W290C), a mutant wherein the aspartic acid residue at position 321 is substituted with alanine (named herein below D321A), a mutant wherein the tyrosine residue at position 340 is substituted with cysteine (named herein below Y340C), a mutant wherein the cysteine residue at position 342 is substituted with arginine (named herein below C342R), a mutant wherein the cysteine residue at position 342 is substituted with serine (named herein below C342S), a mutant wherein the cysteine residue at position 342 is substituted with tryptophan (named herein below C342W), a mutant wherein the asparagine residue at position 549 is substituted with histidine (named herein below N549H), a mutant wherein the lysine residue at position 641 is substituted with arginine (named herein below K641R) in FGFR2. Several severe abnormalities in human skeletal development, including Apert, Crouzon, Jackson-Weiss, Beare-Stevenson cutis gyrata, and Pfeiffer syndromes are associated with the occurrence of mutations in fibroblast growth factor receptor 2. Most, if not all, cases of Pfeiffer Syndrome (PS) are also caused by de novo mutation of the fibroblast growth factor receptor 2 gene, and it was recently shown that mutations in fibroblast growth factor receptor 2 break one of the cardinal rules governing ligand specificity. Namely, two mutant splice forms of fibroblast growth factor receptor, FGFR2c and FGFR2b, have acquired the ability to bind to and be activated by atypical FGF ligands. This loss of ligand specificity leads to aberrant signalling and suggests that the severe phenotypes of these disease syndromes result from ectopic ligand-dependent activation of fibroblast growth factor receptor 2.

In some embodiment, the subject suffers from a FGFR2-related skeletal disease.

As used herein, the term “FGFR2-related skeletal disease” is intended to mean a skeletal disease called craniosynostoses that is caused by an abnormal increased activation of FGFR2, in particular by expression of a constitutively active mutant of the FGFR2 receptor, in particular a constitutively active mutant of the FGFR2 receptor as described above. FGFR2-related skeletal disease relates to Crouzon Syndrome, Jackson-Weiss Syndrome, Apert Syndrome, craniosynostosis, Pfeiffer Syndrome, acrocephalo syndactyly type V, and Beare-Stevenson Cutis Gyrata Syndrome.

As used herein, the term “treatment” or “treat” refer to both prophylactic or preventive treatment as well as curative, improving the patient's condition or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at regular intervals, e.g., daily, weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “preventing” intends characterizing a prophylactic method or process that is aimed at delaying or preventing the onset of a disorder or condition to which such term applies.

The term “expression” when used in the context of expression of a gene or nucleic acid refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein (i.e. FGFR3) produced by translation of a mRNA.

As used herein, the term “catechin” is a flavan-3-ol, a type of natural phenol and antioxidant having the general formula (I):

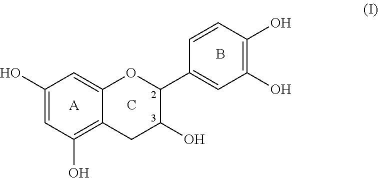

Catechins are plant secondary metabolites and belong to the group of flavan-3-ols (or simply flavanols), part of the chemical family of flavonoids. Catechin possesses two benzene rings (called the A- and B-rings) and a dihydropyran heterocycle (the C-ring) with a hydroxyl group on carbon 3. The A ring is similar to a resorcinol moiety while the B ring is similar to a catechol moiety. There are two chiral centers on the molecule on carbons 2 and 3. Therefore, catechin has four diastereoisomers: two of the isomers are in trans configuration and are called catechin and the other two are in cis configuration and are called epicatechin. The most common catechin isomer is the (+)-catechin. The other stereoisomer is (−)-catechin or ent-catechin. The most common epicatechin isomer is (−)-epicatechin (also known under the names L-epicatechin, epicatechol, (−)-epicatechol, 1-acacatechin, 1-epicatechol, epi-catechin, 2,3-cis-epicatechin or (2R,3R)-(−)-epicatechin). Methods of producing, synthetizing or extracting catechins are well known to the skilled person.

In some embodiment, the catechin isomer is (+)-catechin (2R,3S). As used herein the term “(+)-catechin” has its general meaning in the art and has the following formula:

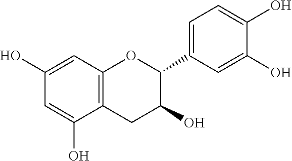 [SMILES: Oc1cc(O)c2c(c1)O[C@H](c1ccc(O)c(O)c1)[C@@H](O)C2]

In some embodiment, the catechin isomer is (−)-catechin (2S,3R). As used herein the term “(−)-catechin” has its general meaning in the art and has the following formula:

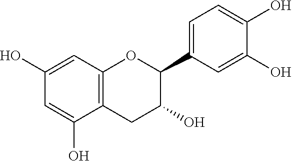 [SMILES: Oc1cc(O)c2c(c1)O[C@@H](c1ccc(O)c(O)c1)[C@H](O)C2]

In some embodiment, the epicatechin isomer is (+)-epicatechin (2S,3S). As used herein the term “(+)-epicatechin” has its general meaning in the art and is having the following formula:

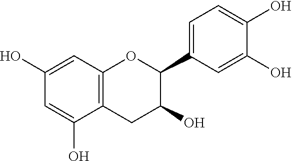 [SMILES: Oc1cc(O)c2c(c1)O[C@@H](c1ccc(O)c(O)c1)[C@@H](O)C2]

In some embodiment, the epicatechin isomer is (−)-epicatechin (2R,3R). As used herein the term “(−)-epicatechin” has its general meaning in the art and refers to (2R,3R)-2-(3,4-dihydroxyphenyl)-3,4-dihydro-2H-chromene-3,5,7-triol and is having the following formula:

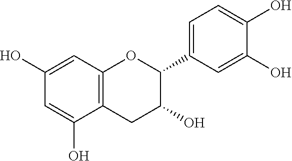 [SMILES: Oc1cc(O)c2c(c1)O[C@H](c1ccc(O)c(O)c1)[C@H](O)C2]

In some embodiment, the (−)-epicatechin is a substantially pure (−)-epicatechin. As used herein, the term “substantially pure” refers to the total absence, or near total absence, of impurities, such as related-substance impurities. For example, when a (−)-epicatechin composition is said to be substantially pure, there are either no detectable related-substance impurities, or if a single related-substance impurity is detected, it is present in an amount no greater than 0.1% by weight, or if multiple related-substance impurities are detected, they are present in aggregate in an amount no greater than 0.6% by weight. The substantially pure (−)-epicatechin according to the invention are capable of inhibiting or eliminating the functional activation of the FGFR3 downstream signalling pathway in vivo and/or in vitro. The a substantially pure (−)-epicatechin may inhibit the functional activation of FGFR3 downstream signalling pathway by at least about 10%, preferably by at least about 20%, preferably by at least about 30%, preferably by at least about 40%, preferably by at least about 50%, preferably by at least about 70, 75 or 80%, still preferably by 85, 90, 95, or 100% or preferably by at least about 300% or preferably by at least about 500%.

In some embodiments, the subject is administered with a pharmaceutical composition comprising the therapeutically effective amount of catechin.

As used herein the terms “administering” or “administration” refer to the act of injecting or otherwise physically delivering a substance as it exists outside the body (e.g. catechin) into the subject, such as by oral (e.g. bottle treatment), mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

A “therapeutically effective amount” refers to an amount effective, at dosages and for periods of time necessary, to achieve a desired therapeutic result. A therapeutically effective amount of drug may vary according to factors such as the disease state, age, sex, and weight of the individual, and the ability of drug to elicit a desired response in the individual. A therapeutically effective amount is also one in which any toxic or detrimental effects of the antibody or antibody portion are outweighed by the therapeutically beneficial effects. The efficient dosages and dosage regimens for drug depend on the disease or condition to be treated and may be determined by the persons skilled in the art. A physician having ordinary skill in the art may readily determine and prescribe the effective amount of the pharmaceutical composition required. For example, the physician could start doses of drug employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. One of ordinary skill in the art would be able to determine such amounts based on such factors as the subject's size, the severity of the subject's symptoms, and the particular composition or route of administration selected. An exemplary, non-limiting range for a therapeutically effective amount of drug is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. As non-limiting examples, treatment according to the present invention may be provided as a daily dosage of the agent of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

In some embodiments, the patient is administered with a pharmaceutical composition comprising the therapeutically effective amount of a catechin as active principle and at least one pharmaceutically acceptable excipient.

As used herein the term “active principle” or “active ingredient” are used interchangeably. As used herein, the term “pharmaceutical composition” refers to a composition described herein, or pharmaceutically acceptable salts thereof, with other agents such as carriers and/or excipients. The pharmaceutical compositions as provided herewith typically include a pharmaceutically acceptable carrier.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remingtons Pharmaceutical-Sciences, Sixteenth Edition, E. W. Martin (Mack Publishing Co., Easton, Pa., 1980) discloses various carriers used in formulating pharmaceutical compositions and known techniques for the preparation thereof.

A pharmaceutically acceptable carrier or excipient refers to a non-toxic solid, semi-solid or liquid filler, diluent, encapsulating material or formulation auxiliary of any type. Typically, the pharmaceutical compositions contain vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Sterile injectable solutions are prepared by incorporating the agent of the present invention in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In some embodiments, the subject is administered with a food composition comprising the therapeutically effective amount of the catechin.

Typically, the food composition comprises or consists of a food product, a food additive, a food ingredient, a functional food, a medical food, a dietary supplement, a nutraceutical, a nutritional supplement or an oral preparation comprising an effective amount of catechin (e.g. bottle treatment). In some embodiments, the food composition is elected from complete food compositions, food supplements, nutraceutical compositions, and the like. The composition of the present invention may be used as a food ingredient and/or feed ingredient. The food ingredient may be in the form of a solution or as a solid-depending on the use and/or the mode of application and/or the mode of administration. The food composition of the present invention may be made using known methods, such as adding an effective amount of the catechin to a food base. The composition that comprises the food product, the food additive, the food ingredient, the functional food, the medical food, the dietary supplement, the nutraceutical, the nutritional supplement or the oral preparation of the present invention may be solid, semi-solid or liquid. It may be in the form of a food product or food supplement, e.g. in the form of tablets, gels, powders, capsules, drinks, bars, etc. For example the composition may be in the form of a powder packed in a sachet which can be dissolved in water, fruit juice, milk or another beverage.

As used herein, the term “food” refers to liquid (i.e. drink), solid or semi-solid dietetic compositions, especially total food compositions (food-replacement), which do not require additional nutrient intake or food supplement compositions. Food supplement compositions do not completely replace nutrient intake by other means. As used herein the term the “food product”, the “food additive”, the “food ingredient” or the “feed ingredient” includes a formulation which is or can be added to functional foods or foodstuffs as a nutritional supplement. By “nutritional food” or “nutraceutical” or “functional food”, is meant a foodstuff which contains ingredients having beneficial effects for health or capable of improving physiological functions. By “food supplement”, is meant a foodstuff having the purpose of completing normal food diet. A food supplement is a concentrated source of nutrients or other substances having a nutritional or physiological effect, when they are taken alone or as a combination in small amounts As used herein the term “functional food” summarizes foodstuff and corresponding products lately developed to which importance is attributed not only due to them being valuable as to nutrition and taste but due to particular ingredient substances. According to the invention, the middle- or long-term maintenance and promotion of health are of importance. In this context, non-therapeutic uses are preferred. The preventive aspect and the promotion of health as well as the food character of the products are, however, best made clear by the term functional food. In many cases, these relate to products accumulated by assortment and selection (as is also the case in the present invention), purification, concentration, increasingly also by addition. Isolated effective substances, in particular in form of tablets or pills, are not included. Although there is no legal definition of a functional food, most of the parties with an interest in this area agree that they are foods marketed as having specific health effects beyond basic nutritional effects. Accordingly, functional foods are ordinary foods that have components or ingredients (such as those described herein) incorporated into them that impart to the food a specific functional e.g. medical or physiological benefit other than a purely nutritional effect.

In some embodiments, the food composition is a drink is a functional drink or a therapeutic drink, a thirst-quencher or an ordinary drink. By way of example, the composition of the present invention can be used as an ingredient to soft drinks, a fruit juice or a beverage comprising whey protein, health teas, cocoa drinks, milk drinks and lactic acid bacteria drinks, yoghurt and drinking yoghurt, cheese, ice cream, water ices and desserts, confectionery, biscuits cakes and cake mixes, snack foods, balanced foods and drinks, fruit fillings, care glaze, chocolate bakery filling, cheese cake flavoured filling, fruit flavoured cake filling, cake and doughnut icing, instant bakery filling creams, fillings for cookies, ready-to-use bakery filling, reduced calorie filling, adult nutritional beverage, acidified soy/juice beverage, aseptic/retorted chocolate drink, bar mixes, beverage powders, calcium fortified soy/plain and chocolate milk, calcium fortified coffee beverage.

The food composition that comprises the food product, the food additive, the food ingredient, the functional food, the medical food, the dietary supplement, the nutraceutical, the nutritional supplement or the oral preparation of the present invention typically comprises carriers or vehicles. “Carriers” or “vehicles” mean materials suitable for administration and include any such material known in the art such as, for example, any liquid, gel, solvent, liquid diluent, solubilizer, or the like, which is non-toxic and which does not interact with any components of the composition in a deleterious manner Examples of nutritionally acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like. In some embodiments, the composition that comprises the food product, the food additive, the food ingredient, the functional food, the medical food, the dietary supplement, the nutraceutical, the nutritional supplement or the oral preparation of the present invention contains protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents, gel forming agents, antioxidants and antimicrobials. The composition may also contain conventional pharmaceutical additives and adjuvants, excipients and diluents, including, but not limited to, water, gelatine of any origin, vegetable gums, ligninsulfonate, talc, sugars, starch, gum arabic, vegetable oils, polyalkylene glycols, flavouring agents, preservatives, stabilizers, emulsifying agents, buffers, lubricants, colorants, wetting agents, fillers, and the like. In all cases, such further components will be selected having regard to their suitability for the intended recipient.

The invention will be further illustrated by the following FIGURES and examples. However, these examples and FIGURES should not be interpreted in any way as limiting the scope of the present invention.

### FIGURES

FIG. 1: (A) MicroCT scans analyses of the mandible at day 14 post fracture. The volume of the callus is unchanged in the treated Fgfr3^{N534K/+} compared to the untreated Fgfr 3^{N534K/+}. The BV/TV of treated Fgfr3^{N534K/+} mandibles is increased. (B) MicroCT scans analyses of the mandible at day 28 post fracture. The volume of the callus is decreased in the treated Fgfr3^{N534K/+} compared to the untreated Fgfr3^{N534K/+}. The BV/TV of treated Fgfr3^{N534K/+} mandibles is increased. (C) Day 28 grade repair. *p<0.05, **p<0.01, ***p<0.001.

### EXAMPLE

### Definitions

As used herein, the term “FGFR3^{N534K/+}” relates to a HCH mouse model. The mutant mice display the clinical features of HCH with growth defects, growth plate anomalies, partial loss of synchondrosis, lordosis. Bone density of the bones of the adults HCH animals is decreased, the bone structure has some characteristics of osteoporotic bones with higher risk of fracture in old age.

As used herein, the term “FGFR3^{Y367C/+}” relates to a mouse model that recapitulates the human ACH phenotype. The clinical hallmarks of ACH (e.g. dwarfism, associated with reduced size of the foramen magnum, hypoplasia of the mandibles, hearing loss, anomalies of the intervertebral discs, defective proliferation and differentiation of the cells of the cartilage and impairment of the ciliogenesis (Pannier et al. 2009, 2010, Mugniery et al 2012, Di Rocco et al 2014, Biosse Duplan et al 2016, Komla Ebri et al 2016, Martin et al 2018).

As used herein, the term “FGFR3^{A385E/+}” relates to a CAN mouse model in which a defective memory was observed.

### Results

I-Study of mandibular bone repair in the mouse model of hypochondroplasia (Fgfr3^{N534K/+}) Mandibular bone repair was studied in the HCH mouse model (Fgfr3^{N534K/+}). The experiments were performed in adult animals at 6 weeks of age, and mice were euthanized at 4 key points: during bone repair (10, 14, 21 days post fracture), and at the end of the normal bone healing process i.e. at 28 days post fracture.

Vertical mandibular fractures were performed in the ascending ramus region in Fgfr3^{N534K/+} and Fgfr3^{+/+} mice. This is a non-stabilized mandibular fracture protocol allowing to analyze the endochondral bone repair process.

Four batches (n=100) of Fgfr3^{N534K/+} and Fgfr3^{+/+} mandible fractures corresponding to different key stages of repair were studied (day 10, day 14, day 21, day 28). Using collagen type II (proliferative cartilage), type X (hypertrophic cartilage) and type I (bone) immunolabelling, we observed that the endochondral process is disturbed, we noted a delayed cartilage resorption, a reduction in the size of hypertrophic chondrocytes area and a defect in bone formation within the repair callus in Fgfr3^{N534K/+} compared to Fgfr3^{+/+}. To complete these data, we performed histomorphometric analyses of the calluses using alcian blue/picrosirus staining at day 28 (post fracture). We observed the presence of pseudarthrosis (fibrosis in the callus) in Fgfr3^{N534K/+} mice whereas we observed 100% of bone in Fgfr3^{+/+} callus. Considering the quality of the newly formed bone at day 28, we defined four grades of repair and classified the mice according to their repair grade (from 1 to 4). The defective repair is significant in Fgfr3^{N534K/+} compared to Fgfr3^{+/+} (Data not shown).

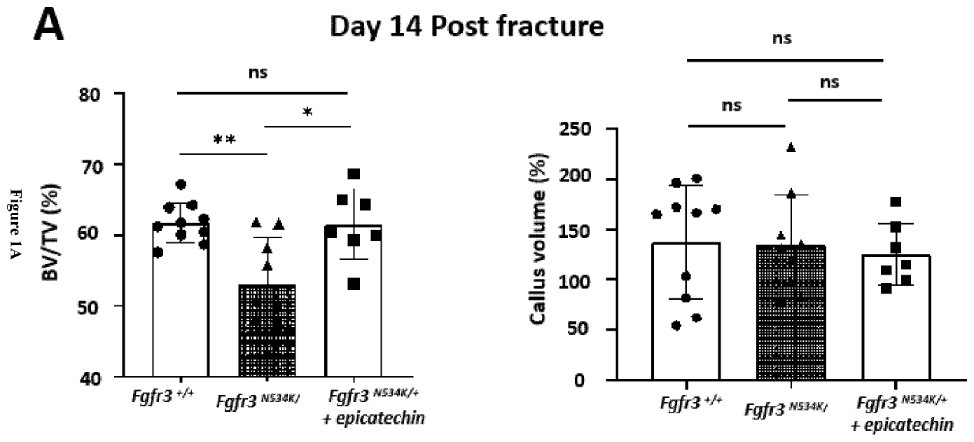

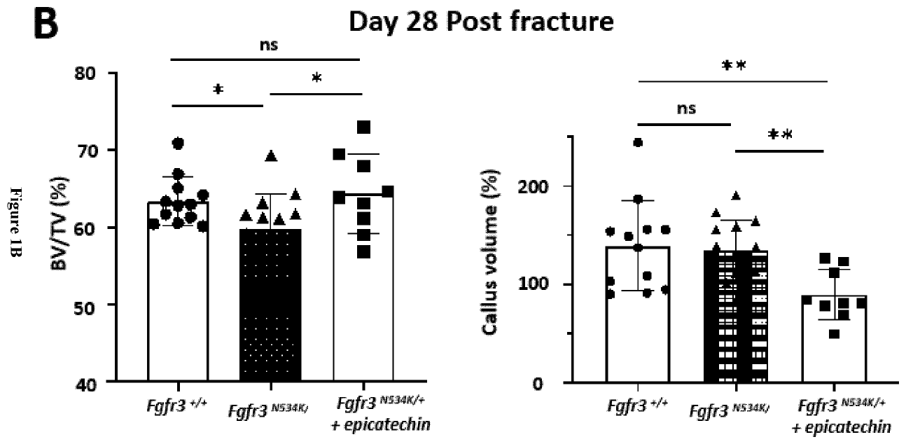

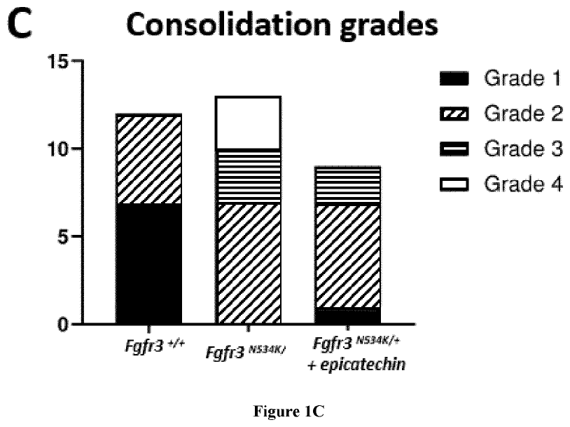

In order to analyze the bone microarchitecture, we performed microCT scans. We observed an alteration in the microarchitecture of callus formation from day 10 to day 28 Fgfr3^{N534K/+} compared to Fgfr3^{+/+}. Bone Volume/Total Volume (BV/TV) are significantly decreased at day 10 (−23%, p<0.05), day 14 (−14%, p<0.01), day 21 (−14.9%, p<0.005), and day 28 (−5.8%, p<0.05) in mutants compared to controls (FIGS. 1A and 1B).

Here, we concluded that Fgfr3 gain of function impaired bone repair and quality in a model of non-stabilized mandibular fracture in HCH mice. We observed that 1) bone formation is altered bone structure has some characteristics of osteoporotic bones and 2) formation of pseudoarthrosis in the callus thus confirming that endochondral ossification is severely disturbed in the mandible.

II-Improvement of Mandible Bone Formation and Repair with (−)-Epicatechin Therapeutic Approach

Current therapeutic approaches to counteract aberrant FGF signaling are many and varied, and include epicatechin targeting signaling pathways downstream of FGFR3 (Martin et al, 2020).

HCH mice (Fgfr3^{N534K/+}) were treated with (−)-epicatechin (subcutaneous injection at 0.3 mg/kg, 3 times per week) from day 0 (facture of the mandible) to day 14 or day 28. The bone repair analyses were evaluated by microCT scans at day 14 (FIG. 1A) and day 28 (FIG. 1B) post fracture.

Significant decrease of the volume of the callus is observed in the treated HCH mice (n=9) compared to untreated mice (n=14) at day 28 post fracture (−39.8%, p<0.01). BV/TV is increased at day 14 post fracture (+19%, p<0.05) in the treated HCH mice (n=7) compared to the untreated HCH mice (n=7) and at day 28 post fracture (+6.3%; p<0.05) in the HCH mice (n=9) compared to the untreated HCH mice (n=14).

FGFR3 gain of function mutation disturbed the bone formation, bone quality and repair in a mandible non-stabilized fracture model. The bone callus is abnormal in HCH mice as revealed by quantitative analyses of the bone by microCT scans. The BV/TV is significantly decreased in HCH mice compared to the wild type.

A comparative analysis of the mandible bone repair of the batch of mice (Fgfr3^{+/+}, Fgfr3^{N534K/+} and Fgfr3^{N534K/+}+ (−)-epicatechin) highlighted the defective bone formation and repair in HCH mice and the benefit effect of Epicatechin treatments in mandible bone repair. Complete bone healing (grade 1) was observed for 1/9. Restoration of bone continuity, but presence of mild bone defects (grade 2) was observed for 6/9 and presence of a bone callus, but with partial bone discontinuity (grade 3) was observed for 2/9 (FIG. 1C). We did not observe pseudarthrosis in treated HCH mice at day 28 post fracture. These results demonstrate the beneficial effect of (−)-epicatechin treatments.

### CONCLUSION

Altogether these data provide the proof of concept that the treatment with (−)-epicatechin improves mandible bone formation and repair in the context of FGFR3-related chondrodysplasia.

### REFERENCES

Throughout this application, various references describe the state of the art to which this invention pertains. The disclosures of these references are hereby incorporated by reference into the present disclosure.

1. A method for the treatment of FGFR-related bone repair and bone formation impairment in a subject in need thereof comprising administering to the subject a therapeutically effective amount of at least one catechin.

2. The method of claim 1 wherein the subject is child or an adult.

3. The method of claim 1 wherein the at least one catechin is (+)-catechin.

4. The method of claim 1 wherein the at least one catechin is (−)-catechin.

5. The method of claim 1 wherein the at least one catechin is (+)-epicatechin.

6. The method of claim 1 wherein the at least one catechin is (−)-epicatechin.

7. The method according to claim 1 wherein the subject harbours a FGFR gain-of-function mutation.

8. The method according to claim 7 wherein the FGFR gain-of-function mutations is a FGFR3-related skeletal disease.

9. The method according to claim 8 wherein the FGFR3-related skeletal disease is hypochondroplasia (HCH), achondroplasia (ACH), thanatophoric dysplasia (TD), Severe Achondroplasia with developmental delay and acanthosis nigricans (SADDAN), Muenke syndrome, Crouzon syndrome with acanthosis nigricans, dwarfism or craniosynostosis.

10. The method according to claim 9 wherein the FGFR3-related skeletal disease is hypochondroplasia (HCH).

11. The method according to claim 9 wherein the FGFR3-related skeletal disease is achondroplasia (ACH).

12. The method according to claim 9 wherein the FGFR3-related skeletal disease is craniosynostosis.

13. The method according to claim 12 wherein the craniosynostosis is Crouzon syndrome with acanthosis nigricans (CAN).

14. The method according to claim 9 wherein the FGFR3-related skeletal disease is Muenke syndrome.

15. The method according to claim 7 wherein the FGFR gain-of-function mutations is a FGFR2-related skeletal disease.

16. The method according to claim 15 wherein the FGFR2-related skeletal disease is Crouzon Syndrome, Jackson-Weiss Syndrome, Apert Syndrome, craniosynostosis, Pfeiffer Syndrome, acrocephalo syndactyly type V, and Beare-Stevenson Cutis Gyrata Syndrome.

17. The method of claim 1 wherein the at least one catechin is administered in a pharmaceutical composition comprising the therapeutically effective amount of the at least one catechin as an active principle and at least one pharmaceutically acceptable excipient.

18. The method of claim 1 wherein the at least one catechin is administered as a food composition comprising the therapeutically effective amount of the at least one catechin.


SEQUENCE LISTING (1 sequence)

SEQ ID NO: 1 - protein, 806 aa, Homo sapiens
MGAPACALALCVAVAIVAGASSESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGILSYGVGFFLFILVVAAVTLCRLRSPPKKGLGSPTVHKISRFPLKRQVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDEYLDLSAPFEQYSPGGQDTPSSSSSGDDSVFAHDLLPPAPPSSGGSRT